(19) United States
(12) Patent Application Publication
(10) Pub. No.: US 2006/0173415 A1
(43) Pub. Date: Aug. 3, 2006

(54) SYRINGE ADAPTOR

(76) Inventor: Christy Cummins, Naas, Co Kildare (IE)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/905,561

(22) Filed: Jan. 11, 2005

(51) Int. Cl. A61M 5/00

(52) U.S. Cl. 604/181

(57) ABSTRACT

A syringe adaptor for use in maintaining a plunger in a fixed position relative to a barrel of a surgical syringe is provided. In one exemplary embodiment, a syringe adaptor is provided having a body that is adapted to mate to a plunger on a syringe, and at least one extension that extends from the body and that is adapted to releasably engage a portion of a barrel on the syringe to hold the plunger in a fixed position relative to the barrel. When the body is mated to a plunger on a syringe, the body can be adapted to move between a first position in which the at least one extension is adapted to engage a portion of the barrel, and a second position in which the body and plunger are freely movable relative to the barrel.

### FIELD OF THE INVENTION

The present invention relates to an adaptor for use with an inflation device, such as a surgical syringe.

### BACKGROUND OF THE INVENTION

Many medical procedures require the use of a balloon catheter. Puncture closure devices, for example, can utilize a balloon catheter to locate the puncture wound and to facilitate positioning of the closure device. Typically, the balloon catheter is inserted through the puncture wound and into the blood vessel with the balloon in a deflated position. A syringe is then filled with fluid and it is connected to a hub on the proximal end of the catheter. The fluid is then injected into the balloon to inflate the balloon with a desired amount of fluid or to a desired pressure. Once the balloon is inflated as desired and positioned at the puncture site, the puncture closure device can then be introduced to close the puncture wound. One problem with current balloon catheters is that once the balloon is inflated, if the user releases the plunger on the syringe, the pressure in the balloon will push the fluid back into the syringe. Consequently, the balloon may pass through the puncture wound during withdrawal of the catheter to locate the puncture.

Accordingly, there remains a need for improved methods and devices to facilitate inflation of a balloon catheter. There also remains a need for improved methods and devices for maintaining a syringe in a fixed position to prevent back flow of fluid into the syringe once fluid is ejected.

### BRIEF SUMMARY OF THE INVENTION

The present invention provides a syringe adaptor for use in maintaining a plunger in a fixed position relative to a barrel of a surgical syringe. The syringe adaptor can be fixedly mated to a surgical syringe, or it can be adapted for use with a variety of surgical syringes. In one exemplary embodiment, a syringe adaptor is provided having a body that is adapted to mate to a plunger on a syringe, and at least one extension that extends from the body and that is adapted to releasably engage a portion of a barrel on the syringe to hold the plunger in a fixed position relative to the barrel. In use, the body can be adapted to move between a first position in which the at least one extension is adapted to engage a portion of the barrel, and a second position in which the body and plunger are freely movable relative to the barrel. In an exemplary embodiment, the body and the plunger can be adapted to rotate relative to the barrel to move between the first and second positions.

While the body can have a variety of configurations, in one exemplary embodiment the body includes first and second extensions extending from opposed sides thereof. Exemplary first and second extensions can have a generally elongate shape with at least one recess formed therein for receiving a flange formed on a proximal end of a barrel on a syringe. In one embodiment, the recess(s) can be formed a distance from a distal end of the first and second extensions. In another embodiment, the first and second extensions can each include multiple recesses such that the body can selectively engage the flange on the barrel in one of several positions to maintain the plunger at a desired position relative to the barrel.

In another exemplary embodiment, a syringe is provided having a barrel with a fluid retaining reservoir formed therein, an outlet formed in a distal end thereof, and a proximal end. A plunger is movably disposed within the fluid retaining reservoir such that the plunger is slidably sealable against an inner surface of the fluid retaining reservoir. The plunger can have a pusher rod attached thereto and extending from a proximal end of the barrel. The syringe can also include a locking element that is fixedly or removably attached to a portion of the pusher rod and that includes at least one extension that is adapted to releasably engage a portion of the barrel to hold the plunger in a fixed position relative to the barrel. In one exemplary embodiment, the extension(s) includes at least one recess formed therein and adapted to releasably engage the proximal end of the barrel. In another exemplary embodiment, each extension can include multiple recesses formed therein for releasably engaging the proximal end of the barrel to selectively maintain the plunger in one of a plurality of positions relative to the barrel. In yet another embodiment, the barrel can include a flange extending around at least a portion of a circumference of the barrel and oriented such that it is substantially transverse to a longitudinal axis of the barrel. The extension(s) can be adapted to releasably engage a portion of the flange.

In yet another exemplary embodiment, a proximal portion of the pusher rod can include a pusher flange at a proximal portion thereof. A connecting portion of the locking element can mate to the pusher flange on the proximal portion of the pusher rod and each extension can be an elongate member that extends from the connecting portion and that is oriented to be substantially parallel to a longitudinal axis of the barrel.

An exemplary method for using a syringe is also provided and includes positioning a syringe adaptor in a first position relative to a plunger on a syringe. A portion of the plunger can be disposed within a barrel of the syringe and the barrel can contain a fluid disposed therein. The plunger is then advanced within the barrel to eject the fluid from the barrel. The syringe adaptor is then rotated to a second position relative to the barrel such that at least one extension on the syringe adaptor engages a portion of the barrel, thereby maintaining the plunger in a fixed position relative to the barrel.

In another exemplary embodiment, the barrel can be coupled to an inflatable balloon, and the balloon can be inflated with fluid by advancing the plunger within the barrel to eject the fluid from the barrel. The syringe adaptor can be rotated relative to the barrel when the balloon is inflated with a predetermined amount of fluid or pressure to maintain the plunger in a fixed position relative to the barrel and to maintain the balloon in the inflated configuration. In another exemplary embodiment, each extension can include one or more recesses formed therein for engaging the flange on the proximal end of the barrel, and each recess can correspond to a predetermined amount of fluid used to inflate the balloon to a certain pressure.

### BRIEF DESCRIPTION OF THE DRAWINGS

The invention will be more fully understood from the following detailed description taken in conjunction with the accompanying drawings, in which:

FIG. 1 is a perspective view of one exemplary embodiment of a syringe adaptor and one exemplary embodiment of a surgical syringe for use with the syringe adaptor;

FIG. 2 is a perspective view of the syringe adaptor shown in FIG. 1;

FIG. 3 is a perspective view of another exemplary embodiment of a syringe adaptor for use with a surgical syringe;

FIG. 4A is a perspective view of the syringe adaptor shown in FIGS. 1 and 2 coupled to the surgical syringe shown in FIG. 1, showing the step of filling the syringe with fluid;

FIG. 4B is a perspective view of the syringe adaptor and surgical syringe shown in FIG. 4A mated to a balloon catheter;

FIG. 5A is a perspective view of the syringe adaptor and surgical syringe shown in FIG. 4A showing the step of ejecting fluid from the syringe;

FIG. 5B is a perspective view of the syringe adaptor, surgical syringe, and balloon catheter shown in FIG. 4B showing the step of ejecting fluid from the syringe to inflate the balloon;

FIG. 6A is a perspective view of the syringe adaptor and surgical syringe shown in FIG. 5A showing the step of rotating the syringe adaptor to lock the surgical syringe in a fixed position;

FIG. 6B is a perspective view of the syringe adaptor, surgical syringe, and balloon catheter shown in FIG. 5B showing the step of rotating the syringe adaptor to lock the surgical syringe in a fixed position and thereby maintain the balloon in the inflated configuration;

FIG. 7 is a perspective view of the syringe adaptor and surgical syringe in FIG. 6A shown in the locked position, and showing the step of rotating the syringe adaptor to unlock the surgical syringe; and

FIG. 8 is a perspective view of the syringe adaptor and surgical syringe shown in FIG. 7, showing the step of withdrawing fluid from the catheter and balloon and refilling the syringe with fluid.

### DETAILED DESCRIPTION OF THE INVENTION

The present invention provides a syringe adaptor that is effective to maintain a plunger of a surgical syringe in a fixed position relative to a barrel of the syringe. The syringe adaptor can be formed integrally with or fixedly mated to a surgical syringe, or it can be removably matable to a surgical syringe such that it can be used with a variety of syringes. In one exemplary embodiment, the syringe adaptor can be used with a surgical syringe to inflate a balloon on a balloon catheter, and to maintain the balloon in an inflated configuration during a surgical procedure. A person skilled in the art will appreciate that, while the syringe adaptor is described in connection with a procedure that utilizes a balloon catheter, the syringe adaptor can be used for a variety of medical purposes in which it may be necessary to maintain a plunger of a syringe in a fixed position relative to the barrel.

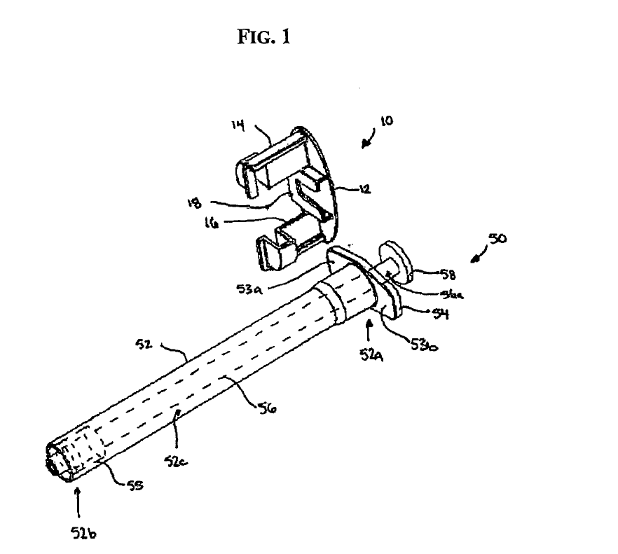

FIG. 1 illustrates one exemplary embodiment of a syringe adaptor 10 and a surgical syringe 50. While the syringe adaptor 10 can be used with a variety of syringes, for reference purposes the syringe 50 shown in FIG. 1 is a standard surgical syringe. As shown, the syringe 50 includes a barrel 52 having a distal end 52b that is adapted to release fluid therefrom, a proximal end 52a with a flange 54 formed thereon, and a fluid retaining reservoir 52c formed therein for retaining fluid. In an exemplary embodiment, as shown, the flange 54 formed on the proximal end 52a of the barrel 52 has a generally elongate, somewhat rectangular shape such that it includes opposed tabs 53a, 53b that extend from opposed sides of the proximal end 52a of the barrel 52. The syringe 50 also includes a plunger 55 that is sealingly and slidably disposed within the barrel 52, and a pusher rod 56 that is attached to the plunger 55 for moving the plunger 55 relative to the barrel 52. In an exemplary embodiment, a proximal end 56a of the pusher rod 56 includes a substantially circular, planar pusher flange 58 formed thereon to facilitate grasping of the pusher rod 56 and to facilitate mating to the syringe adaptor 10. In use, as the plunger 55 is moved proximally fluid is drawn into the fluid retaining reservoir 52c in the barrel 52, and as the plunger 55 is moved distally fluid is ejected from the fluid retaining reservoir 52c.

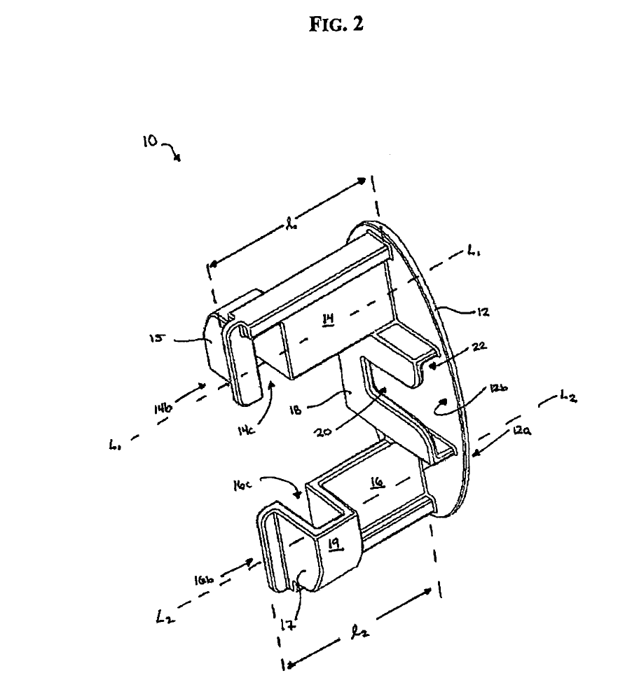

As noted above, the syringe adaptor 10 is configured to mate to the syringe 50 to maintain the plunger 55 in a fixed position relative to the barrel 52. While the syringe adaptor 10 can have a variety of configurations, the exemplary syringe adaptor 10, which is shown in more detail in FIG. 2, generally includes a body portion 12 that is adapted to be coupled to or fixedly mate to the pusher rod 56 of the surgical syringe 50, and at least one locking element, such as an extension 14, 16, that extends from the body 12 and that is adapted to releasably engage a portion of the barrel 52 of the syringe 50 to hold the plunger 55 and pusher rod 56 in a fixed position relative to the barrel 52.

The body 12 of the syringe adaptor 10 can have a variety of configurations, but in the illustrated exemplary embodiment the body 12 has a generally circular planar configuration with a proximal surface 12a and a distal surface 12b. A mating element 18 is formed on the distal surface 12b of the body 12 and it is adapted to removably mate to the pusher rod 56 of the surgical syringe 50. While virtually any mating element 18 can be used, in the illustrated exemplary embodiment the mating element 18 is in the form of a housing having a slot 20 extending therethrough for receiving the rod-portion of the pusher rod 56, and a channel 22 formed therein for receiving the flange 58 formed on the proximal end 56a of the pusher rod 56. The mating element 18 can also be adapted to create an interference fit, snap-fit, or other engaging fit with the flange 58 to prevent accidental removal of the syringe adaptor 10 during use of the device. A force can be used to overcome the interference fit, snap-fit, or other engaging fit to remove the syringe adaptor 10 from the syringe 50. The syringe adaptor 10 can alternatively be fixedly mated to the flange 58 on the pusher rod 56 using, for example, adhesive, RF welding, a mechanical latch, or other mating techniques. The syringe adaptor 10 can also alternatively be integrally formed with the surgical syringe 50. A person skilled in the art will appreciate that a variety of other techniques can be used to mate the syringe adaptor 10 to the pusher rod 56 on the surgical syringe 50, and that the mating element 18 can have a variety of other configurations depending on the configuration of the surgical syringe.

As noted above, the syringe adaptor 10 can also include a locking element, such as an extension 14, 16, that extends from the body 12 and that is adapted to releasably engage a portion of the barrel 52 of the syringe 50. As shown in FIG. 2, the syringe adaptor 10 includes first and second extensions 14, 16 that extend from opposed sides of the distal surface 12b of the body 12. The extensions 14, 16 can vary in shape and size, but in the illustrate exemplary embodiment the first and second extensions 14, 16 each have a generally elongate shape with a distal end 14b, 16b that is adapted to engage the flange 54 formed on the proximal end 52a of the barrel 52 of the surgical syringe 50. In particular, the distal end 14b, 16b of each extension includes a recess 14c, 16c formed therein for receiving the flange 54 formed on the proximal end 52a of the barrel 52. Each recess 14c, 16c can vary in shape and size depending on the shape and size of the flange 58 on the barrel 52 of the syringe 50. As shown in FIG. 2, the recesses 14c, 16c are each formed in a housing 15, 17 that is oriented substantially transverse to a longitudinal axis L1, L2 of the extension 14, 16. The recesses 14c, 16c can extend completely through the housing 15, 17 to allow the flange 54 to pass completely therethrough when the syringe adaptor 10 is rotated relative to the barrel 52. Alternatively, each recess 14c, 16c can extend only partially through the housing 15, 17, as shown, such that the housing 15, 17 includes a stop surface (only one stop surface 19 is shown in FIG. 2). With this configuration, the housings 15, 17 preferably extend in opposite directions from one other, as shown. This will allow the syringe adaptor 10 to be rotated in a first direction, in which the recesses 14c, 16c receive the flange 54 on the barrel 52, and in a second, opposite direction, in which the recesses 14c, 16c are removed from the flange 54 to thereby allow free movement of the plunger 55 and pusher rod 56 relative to the barrel 52, as will be discussed in more detail below.

The length l₁, l₂ of each extension 14, 16 and/or the location of each recess 14c, 16c can also vary, but in an exemplary embodiment the length l₁, l₂ is adapted to allow the recesses 14c, 16c on the extensions 14, 16 to engage the flange 58 on the barrel 52 when the plunger 55 and pusher rod 56 are at a predetermined position relative to the barrel 52. For example, the extensions 14, 16 can have a length l₁, l₂ that is configured such that the recesses 14c, 16c will be positioned to engage the flange 54 when the plunger 55 and pusher rod 56 are fully disposed within the barrel 52. In this position, all fluid will be ejected from the barrel 52.

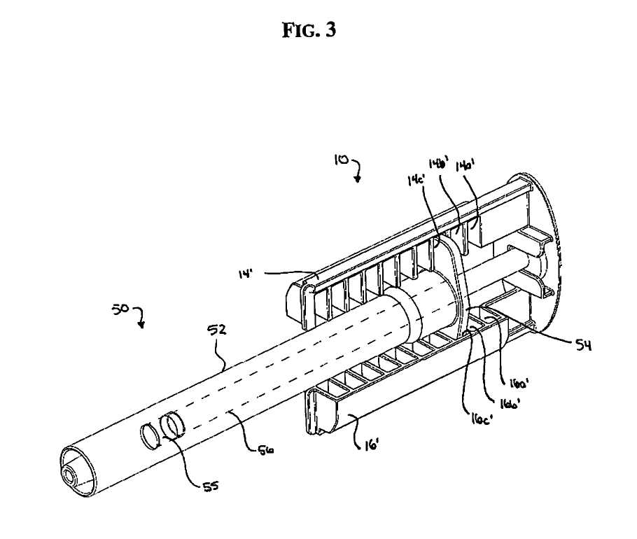

In another embodiment, shown in FIG. 3, each extension 14′, 16′ can include several recesses 14a′, 16a′, 14b′, 16b′, 14c′, 16c′, etc. formed therein along the length thereof to allow the plunger 55 and pusher rod 56 to be maintained in a selected position relative to the barrel 52. As shown in FIG. 3, the flange 54 on the barrel 52 is positioned within recesses 14c′, 16c′ such that the plunger 55 is maintained at a location proximal to the distal end of the barrel 52. Such a configuration allows a user to eject a selected amount of fluid and to maintain the plunger 55 and pusher rod 56 at a desired position that corresponds to the ejected amount of fluid. A person skilled in the art will appreciate that a variety of other techniques can be used to allow the plunger 55 to be maintained at a selected position.

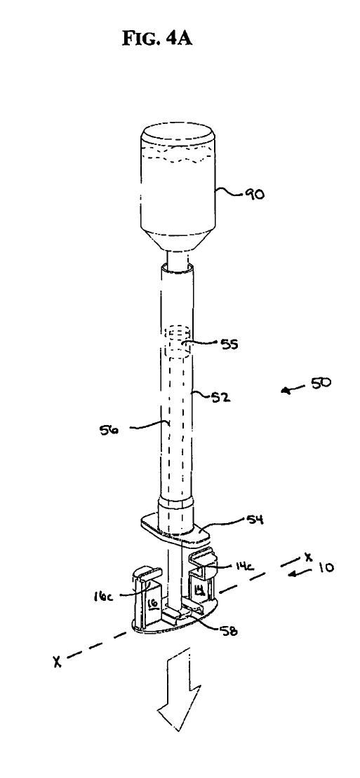

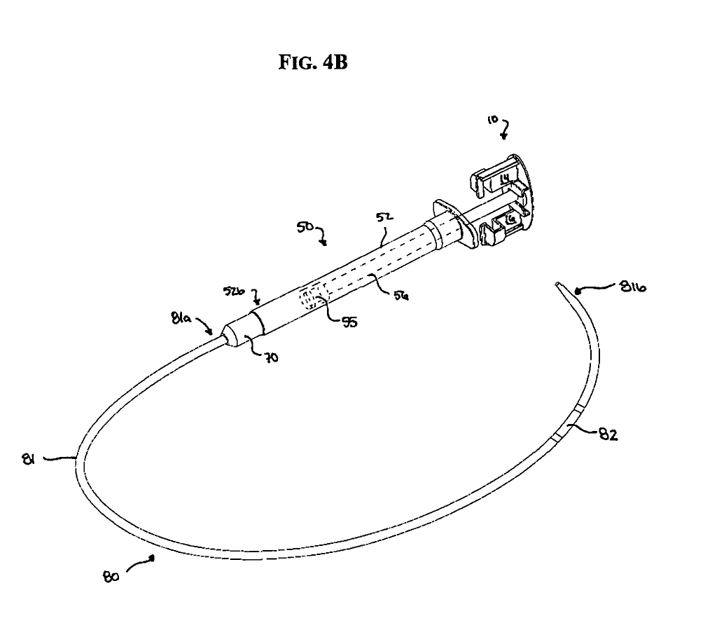

As previously noted, in an exemplary embodiment the syringe adaptor 10 and syringe 50 can be adapted for use with a balloon catheter. In particular, the syringe 50 can be used to inflate a balloon with an inflation fluid, and the syringe adaptor 50 can be used to maintain the plunger 55 and pusher rod 56 in a fixed position relative to the barrel 52 to thereby maintain the balloon in the inflated configuration. A medical procedure can then be performed and once completed the syringe adaptor 10 can be unlocked to release the pusher rod 56 and thereby allow the inflation fluid to be removed from the balloon. FIGS. 4A-8 illustrate the syringe adaptor 10 used in combination with surgical syringe 50 and with an exemplary balloon catheter 80.

As shown in FIG. 4A, the syringe adaptor 10 is coupled to the flange 58 on the pusher rod 56. When first connected, the extensions 14, 16 are preferably positioned such that they are offset from the flange 54 to allow the adaptor 10 to move proximally and distally relative to the syringe 50 without the extensions 14, 16 coming into contact with the flange 54. In other words, the extensions 14, 16 reside in a plane that is substantially perpendicular to an axis X of the flange 54. A person skilled in the art will understand that the syringe adaptor 10 can be connected to the syringe 10 at any step during the procedure.

The barrel 52 of the syringe 10 is then filled with an inflation fluid, such as saline, by pulling the syringe adaptor 10 and pusher rod 56 in a proximal direction, as indicated by arrow P. As a result, the plunger 55 is moved proximally within the barrel 52, thereby drawing fluid into the barrel 52 from a fluid source 90. The pusher rod 56 and plunger 55 can be moved proximally P by an amount that is sufficient to fill the barrel 52 with a desired amount of fluid. A person skilled in the art will appreciate that standard techniques known in the art can be used to connect the distal end of the syringe 10 to a fluid source 90.

Once the barrel 52 is filled with a desired amount of fluid, the syringe 10 can be coupled to a balloon catheter 80, as shown in FIG. 4B. The balloon catheter 80 can be positioned within a patient's body either prior to or after coupling the syringe 10 thereto. As shown, the balloon catheter 80 generally includes an elongate catheter 81 having a proximal end 81a that is coupled to a hub 70 connected to the distal end 52b of the barrel 52 of the syringe 10, and a distal end 81b with a balloon 82 formed thereon. In the embodiment shown in FIG. 4B, the balloon 82 is positioned proximal to the distal end 81b. A person skilled in the art will understand that the balloon catheter 80 can have virtually any configuration and that balloon catheter 80 is merely shown for illustration purposes.

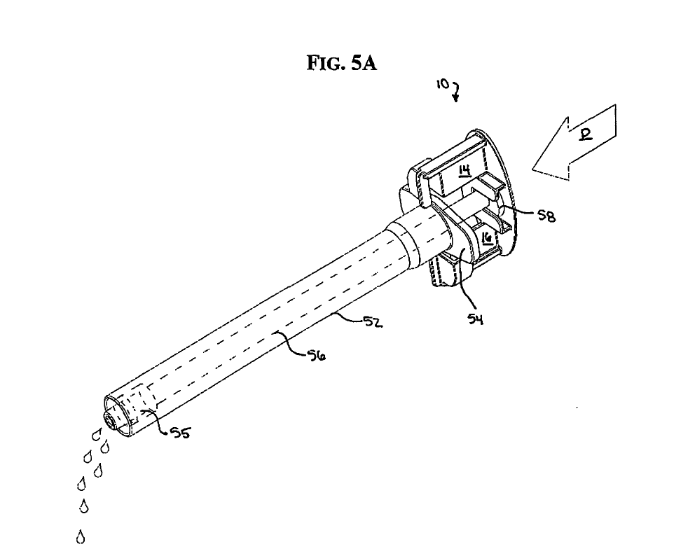

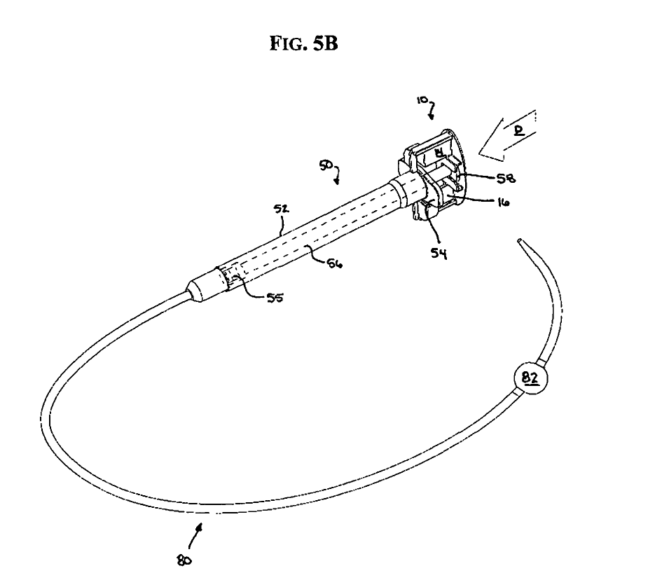

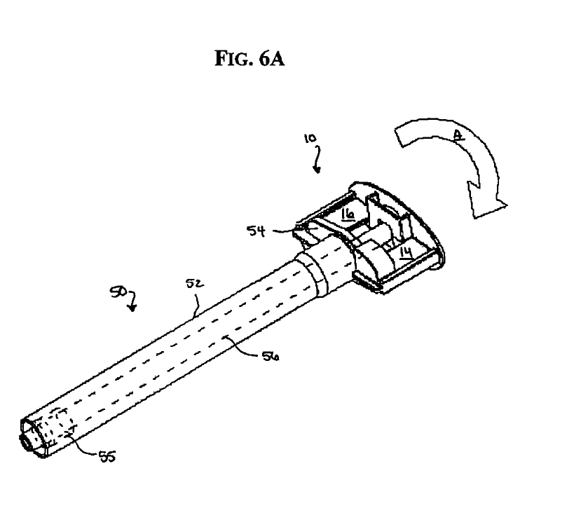

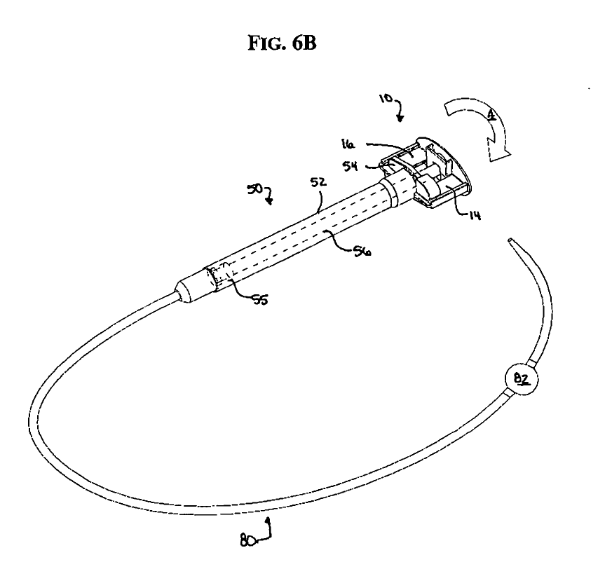

The syringe adaptor 10 is then moved in a distal direction, as indicated by arrow D in FIGS. 5A and 5B, to eject fluid from the barrel 52 into the balloon catheter 80, thereby inflating the balloon 82, as shown in FIG. 5B. A pressure regulator device (not shown) may be positioned between the distal end 52b of the barrel 52 and a proximal end 81a of the catheter 81. As fluid is injected into the balloon 82, the regulator will indicate visually when a certain internal pressure has been reached within the balloon 82. Once the balloon 82 is inflated to a desired size and/or pressure, the balloon 82 can be maintained at the desired configuration by rotating the syringe adaptor 10 in a first direction, as indicated by arrow A in FIGS. 6A and 6B, to cause the extensions 14, 16 to engage the flange 54 on the proximal end of the pusher rod 56. In particular, as the syringe adaptor 10, and consequently the pusher rod 56 and plunger 55, are rotated the recesses 14c, 16c will receive the opposed tabs 53a, 53b of the flange 54. The syringe adaptor 10 will thereby prevent movement of the pusher rod 56 and plunger 55 relative to the barrel 52, thereby preventing backflow of fluid from the balloon 82 into the barrel 52 and forward flow of fluid into the balloon 82 increasing its inflated diameter and pressure. A surgical procedure can then be performed.

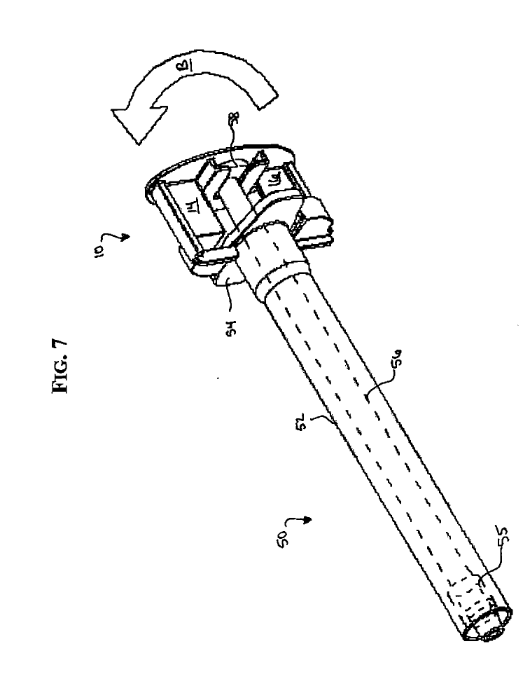

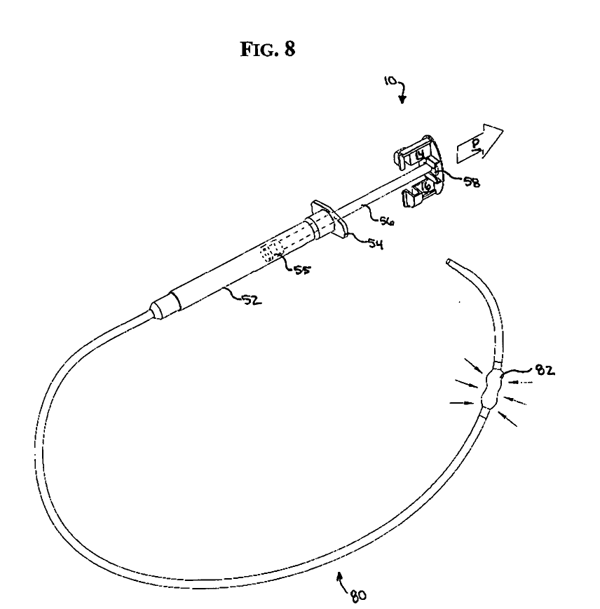

Once the procedure is complete, the balloon 82 can be deflated by rotating the syringe adaptor 10 in a second, opposite direction, as indicated by arrow B in FIG. 7, to relative the flange 54. The pusher rod 56 and plunger 55 can then be moved proximally P, as shown in FIG. 8, to draw fluid from the balloon 82 back into the barrel 52.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A syringe adaptor, comprising:
a body that is adapted to mate to a plunger on a syringe, and at least one extension that extends from the body and that is adapted to releasably engage a portion of a barrel on the syringe to hold the plunger in a fixed position relative to the barrel.

2. The syringe adaptor of claim 1, wherein, when the body is mated to a plunger on a syringe, the body is adapted to move between a first position in which the at least one extension is adapted to engage a portion of the barrel, and a second position in which the body and plunger are freely movable relative to the barrel.

3. The syringe adaptor of claim 2, wherein, when the body is mated to a plunger and in the first position, the body is adapted to position the plunger substantially fully disposed within the barrel.

4. The syringe adaptor of claim 2, wherein, when the body is mated to a plunger, the body and the plunger are adapted to rotate relative to the barrel.

5. The syringe adaptor of claim 1, wherein the body includes first and second extensions extending from opposed sides thereof.

6. The syringe adaptor of claim 5, wherein the first and second extensions have a generally elongate shape with at least one recess formed therein for receiving a flange formed on a proximal end of a barrel on a syringe.

7. The syringe adaptor of claim 6, wherein the at least one recess is formed a distance from a distal end of the first and second extensions.

8. The syringe adaptor of claim 6, wherein the first and second extensions each include a plurality of recesses such that the body is adapted to maintain a plunger on a syringe in a plurality of positions relative to a barrel of a syringe.

9. A syringe, comprising:
a barrel adapted to retain fluid therein;
a plunger movably disposed within the barrel and slidably sealable with an inner surface of the barrel, the plunger having a pusher rod attached thereto and extending from a proximal end of the barrel; and
a locking element attached to a portion of the pusher rod, the locking element having at least one extension that is adapted to releasably engage a portion of the barrel to hold the plunger in a fixed position relative to the barrel.

10. The syringe of claim 9, wherein the at least one extension includes at least one recess formed therein and adapted to releasably engage the proximal end of the barrel.

11. The syringe of claim 9, wherein the locking element includes first and second elongate extensions, and wherein each extension includes a plurality of recesses formed therein for releasably engaging the proximal end of the barrel to maintain the plunger in one of a plurality of positions relative to the barrel.

12. The syringe of claim 9, wherein the locking element is removably attached to the plunger.

13. The syringe of claim 9, wherein the barrel includes a flange, the flange extending around at least a portion of a circumference of the barrel and being oriented such that it is substantially transverse to a longitudinal axis of the barrel, and wherein the at least one extension is adapted to releasably engage a portion of the flange.

14. The syringe of claim 9, wherein a proximal portion of the pusher rod includes a pusher flange formed thereon, and wherein the locking element is adapted to removably engage the pusher flange.

15. The syringe of claim 9, wherein a proximal portion of the pusher rod includes a pusher flange formed thereon, and wherein the locking element is fixedly attached to the pusher flange.

16. The syringe of claim 14, wherein the locking element includes a body having a mating element formed thereon and adapted to mate to the pusher flange on the proximal portion of the pusher rod, and wherein the at least one extension comprises an elongate member that extends from the body and that is oriented to be substantially parallel to a longitudinal axis of the barrel.

17. A method for using a syringe comprising:
positioning a syringe adaptor in a first position relative to a plunger on a syringe, a portion of the plunger being disposed within a barrel of the syringe and the barrel containing a fluid disposed therein;
advancing the plunger within the barrel to eject the fluid from the barrel;
rotating the syringe adaptor to a second position relative to the barrel such that at least one extension on the syringe adaptor engages a flange formed on a proximal end of the barrel, thereby maintaining the plunger in a fixed position relative to the barrel.

18. The method of claim 17, wherein the barrel is coupled to an inflatable balloon, and wherein advancing the plunger within the barrel to eject the fluid from the barrel comprises inflating the balloon with the fluid.

19. The method of claim 18, wherein the syringe adaptor is rotated relative to the barrel when the balloon is inflated with a predetermined amount of fluid to maintain the plunger in a fixed position relative to the barrel and to maintain the balloon in the inflated configuration.

20. The method of claim 19, wherein the at least one extension includes a plurality of recesses formed therein for engaging the flange on the proximal end of the barrel, each recess corresponding to a predetermined amount of fluid used to inflate the balloon.

21. The method of claim 17, wherein the at least one extension includes at least one recess formed therein for engaging the flange on the proximal end of the barrel.
